(19) United States
(12) Patent Application Publication
(10) Pub. No.: US 2008/0021099 A1
(43) Pub. Date: Jan. 24, 2008

(54) 5-Cyano-prostacyclin derivatives as agents for the treatment of autoimmune disease

(75) Inventors: Daryl Faulds, Mill Valley, CA (US); William Guilford, Belmont, CA (US); Judy Li, Emeryville, CA (US)

Correspondence Address: JACQUELINE S LARSON, 245 AVINGTON ROAD, ALAMEDA, CA, 94502, US

(73) Assignee: Bayer Schering Pharma Aktiengesellschaft, Berlin (DE)

(21) Appl. No.: 11/879,076

(22) Filed: Jul. 16, 2007

Related U.S. Application Data
(60) Provisional application No. 60/831,801, filed on Jul. 18, 2006.

(51) Int. Cl. A61K 31/343; A61P 37/00

(52) U.S. Cl. 514/469

(57) ABSTRACT

The present invention is directed to the use of 5-cyano-prostacyclin derivatives as therapeutics for the treatment of autoimmune diseases.

The present invention is directed to the use of 5-cyano-prostacyclin derivatives as therapeutics for the treatment of autoimmune diseases.

### BACKGROUND OF THE INVENTION

The effects of prostaglandins are mediated by their G protein-coupled receptors which are located on the cell surface. Prostaglandin E₂ (PGE₂) is of particular interest, having a wide variety of cellular effects through binding to functionally different receptor subtypes, namely the EP1, EP2, EP3 and EP4 receptors.

Cytokine production by mature antigen-carrying dendritic cells (DC) within lymph nodes is strongly influenced by PGE₂ during their activation in peripheral tissues. Inflammatory cytokines such as IL-1 and TNF-α activate antigen-carrying DC to secrete IL-12 and promote the development of T-helper type 1 (Th-1) cytokine expression-biased cells. In contrast, DC activated in the presence of PGE₂ show impaired IL-12 production and promote the development of T-helper type 2 (Th-2) cytokine expression-biased cells [Hilkens C M et al., J. Immunol. 156:1722-27 (1996)]. The difference in the ability to produce IL-12 in response to PGE₂, established during DC activation in the peripheral tissues, is stable to the removal of cytokines and PGE₂.

Peripheral blood mononuclear cells (PBMC) obtained from multiple sclerosis (MS) patients, although responsive to PGE₂, require higher levels of the prostaglandin to achieve beneficial responses equivalent to those seen in PBMC obtained from healthy control donors. Dore-Duffy et al. [E. Clin Immunol Immunopathol. 61:119-128 (1990)] studied the functional response of monocytes from MS patients. They found that MS monocytes were less sensitive to PGE₂-mediated increases in cAMP (acting through EP2 or EP4). These observations suggest that MS patients require higher levels of PGE₂ to elicit beneficial immunomodulatory responses.

Ruddle et al. first showed that TNF-α and the T cells that produce it play important roles in central nervous system autoimmune disease [Ruddle N H, et al., J Exp Med. 172(4):1193-200 (1990)]. It has also been shown that a combination of IL-2, GM-CSF and MIP1α have potent immuno-stimulatory properties [Zilbert A., et al., Hum Gene Ther. 15(1):21-34 (2004)]. On the other hand, IL-15 has been shown to have an opposing effect of IL-2 on T cells [Lee J M, et al., Immunol. 173(5):3155-64 (2004)]. Additionally, since administration of IFN-γ is known to promote exacerbations of MS [Panitch H S, et al., J Neuroimmunol. 46(1-2):155-64 (1993)], it would be expected that reduction of Th-1 cytokine expression, such as IFN-γ, while sparing Th-2 cytokine expression, such as IL-4, would be of benefit to MS patients.

In addition, CD8⁺ T cells have been found to dominate the T cell infiltrate in active MS lesions, and it has been speculated that these cells are actively involved in the disease process [Babbe H et al. J Exp Med. 192:393-404 (2000)].

It would therefore be desirable in treating MS, as well as other autoimmune diseases, to have a therapeutic agent that inhibits the release of Th-1 cytokines while sparing the expression of Th-2 cytokines, and that enhances a polarization of T cells recruitment towards the Th-2 response and away from the Th-1 response. A therapeutic agent that impairs the CD8⁺ cytotoxic T cell response would also be desirable for treating autoimmune diseases.

### SUMMARY OF THE INVENTION

The present invention is directed to agents that are useful as therapeutics for autoimmune diseases. More particularly, the invention is directed to a method of treating autoimmune diseases by administering to a patient in need thereof a therapeutically effective amount of a 5-cyano-prostacyclin derivative.

Autoimmune diseases that may be treated according to the present invention include, but are not limited to, multiple sclerosis (MS), secondary progressive multiple sclerosis (SPMS), psoriasis, rheumatoid arthritis, Crohn's disease, and alopecia areata.

### DETAILED DESCRIPTION OF THE INVENTION

5-Cyano-prostacyclin derivatives useful in the present invention are those that inhibit the release of one or more Th-1 cytokines (such as, for example, IL-2, IL-12, IFN-γ, and GM-CSF) while sparing the expression of Th-2 cytokines (such as, for example, IL-4 and IL-10).

5-Cyano-prostacyclin derivatives and certain of their pharmacological effects are known from U.S. Pat. Nos. 4,219,479 and 4,049,582, the entire disclosures of which are incorporated herein by reference. These compounds have been found to be effective EP2 and EP4 agonists. The production of these compounds and the pharmaceutically acceptable salts thereof are described in detail in the above U.S. patents. Cyclodextrin clathrates of the 5-cyano-prostacyclin derivatives are also included within the scope of the present invention; they are disclosed and claimed in U.S. Pat. No. 5,010,065, the entire disclosure of which is incorporated herein by reference. The above 5-cyano-prostacyclin derivatives have not been previously disclosed as being effective in the treatment or prevention of multiple sclerosis or other autoimmune diseases, and this new pharmacological property also has no direct connection with the effects described in the U.S. patents.

In one embodiment, the 5-cyano-prostacyclin derivative useful in treating autoimmune diseases according to the present invention is Nileprost (5-cyano-15-methylprostacyclin):

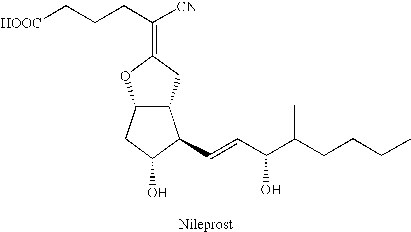

It has now been found that the above-described 5-cyano-prostacyclin derivatives inhibit the release of Th-1 cytokines while sparing the expression of Th-2 cytokines and enhance a polarization of T cells recruitment towards the Th-2 response and away from the Th-1 response. This unexpected and surprising activity makes them desirable as pharmaceuticals for treating MS and other autoimmune diseases. This is particularly true since they are distinguished over natural prostaglandins by an improved specificity, longer period of effectiveness and higher stability. Additionally, these compounds have been found in clinical phase I studies to be very well-tolerated by humans and to have no hypotensive effects, making them further suitable as pharmaceuticals.

The pharmacologically active 5-cyano-prostacyclin derivatives above can be processed in accordance with conventional methods of galenic pharmacy to produce medicinal agents for treating autoimmune diseases, such as (but not limited to) multiple sclerosis, secondary progressive multiple sclerosis, psoriasis, rheumatoid arthritis, Crohn's disease, and alopecia areata. The pharmaceutical compositions comprise the 5-cyano-prostacyclin derivative in a therapeutically effective amount (that is, an amount effective to treat an autoimmune disease) and one or more pharmaceutically acceptable excipients. Suitable excipients may include, but are not limited to, pharmaceutical, organic or inorganic inert carrier materials suitable for enteral, parenteral or topical administration which do not deleteriously react with the active compounds. Suitable pharmaceutically acceptable carriers include, but are not limited to, water, salt solutions, alcohols, gelatine, gum arabic, lactate, starch, magnesium stearate, talc, vegetable oils, polyalkylene glycols, polyvinyl pyrrolidone, hydroxyl-methylcellulose, silicic acid, viscous paraffin, fatty acid monoglycerides and diglycerides, and the like. The pharmaceutical products may be in solid form, for example as tablets, coated tablets, suppositories or capsules, or in liquid form, for example as solutions, suspensions or emulsions. They may additionally comprise, where appropriate, auxiliary agents such as lubricants, preservatives, stabilizers, wetting agents, emulsifiers, salts to alter the osmotic pressure, buffers, coloring, flavoring, and/or aromatic substances and the like that do not deleteriously react with the active compounds. Examples of suitable pharmaceutical compositions include the following:

Particularly suitable for oral use are tablets, coated tablets or capsules with talc and/or carbohydrate carriers or binders, such as, for example, lactose, maize starch or potato starch. Use is also possible in liquid form, such as, for example, as fluid to which a sweetener is added where appropriate.

Sterile, injectable, aqueous or oily solutions are used for parenteral administration, as well as suspensions, emulsions or implants, including suppositories. Ampoules are convenient unit dosages. Sustained release compositions can be formulated including those wherein the active compound is protected with differentially degradable coatings, e.g., by microencapsulation, multiple coatings, etc.

Carrier systems which can also be used are surface-active excipients such as salts of bile acids or animal or vegetable phospholipids, but also mixtures thereof, and liposomes or constituents thereof. Transdermal patches may also be used as delivery means.

The dosage of the 5-cyano-prostacyclin derivatives will be that amount effective to treat an autoimmune disease. The effective amount of active ingredient may vary depending on the route of administration, the age and weight of the patient, the nature and severity of the disorder to be treated, and similar factors. The effective amount can be determined without undue experimentation by methods known to those of skill in the art. The daily dose is generally about 0.1-200 μg/kg/day, preferably about 0.5-10 μg/kg/day, when administered to human patients, it being possible for the dose to be given as a single dose to be administered once or divided into two or more doses administered daily.

Without further elaboration, it is believed that one skilled in the art can, using the preceding description, utilize the present invention to its fullest extent. The following specific embodiments are, therefore, to be construed as merely illustrative, and not limitative of the remainder of the disclosure in any way whatsoever.

### EXAMPLES

### Example 1

### Demonstration of Nileprost Inhibiton of T Lymphocyte Th-1 Cytokine Release

### Principle:

The activation of a human T lymphocyte by an antigen-presenting cell and antigen through the T cell receptor is mimicked in experimental conditions by the lectin Concanavilin A (ConA). It is known that ConA binds to the T cell receptor and stimulates the cell to release various cytokines. The binding of Nileprost to the EP receptor inhibits various cytokines release. One of the Th-1 released cytokines is IFN-γ. The biochemistry and biological activities of IFN-γ have been extensively reviewed in the literature.

### Detection Method:

Human IFN-γ is a dimer of the expressed 143 amino acid protein. Enzyme-linked immunosorbant assays (ELISA) based on antibodies specific to IFN-γ are commercially available. Standards and samples are pipetted into the wells of a microplate. An antibody specific to human IFN-γ is added to the wells. A substrate is added to the wells and color develops in proportion to the amount of IFN-γ bound. The intensity of the color is measured.

### Procedure:

Peripheral blood lymphocytes were isolated from human donors using a Ficoll density gradient and residual erythrocytes were removed by selective lysis. The lymphocytes are cultured at approximately 10⁶ cells per mL in RPMI1640 with 10% additional fetal bovine serum. The cell cultures were activated with 2 μg/mL of ConA as described above. Nileprost was added at various dilutions during the ConA activation. Cells were incubated for approximately 18 hr at 37° C. IFN-γ released during activation was measured by ELISA.

Nileprost Inhibition (percent)
human T cells
ConA stimulation
IFN-γ (pg/mL)

| | Nileprost (nM) | | | | | | |
|---|---|---|---|---|---|---|---|
| | 0 | 0.5 | 1.25 | 2.5 | 5 | 10 | 20 |
| Donor 1 | 0% | 17% | 23% | 45% | 56% | 50% | 67% |
| standard deviation | 21% | 21% | 3% | 11% | 10% | 10% | 5% |
| Donor 2 | 0% | 2% | 11% | 17% | 35% | 43% | 41% |
| standard deviation | 17% | 29% | 21% | 19% | 6% | 10% | 7% |
| Donor 3 | 0% | 26% | 36% | 43% | 54% | 64% | 68% |
| standard deviation | 12% | 9% | 2% | 3% | 2% | 7% | 10% |
| Donor 4 | 0% | 21% | 29% | 31% | 48% | 55% | 56% |
| standard deviation | 13% | 6% | 12% | 8% | 8% | 5% | 6% |

### Nileprost showed, dose responsively, very high inhibitory activity in the T lymphocyte Th-1 cytokine release.

### Example 2

### Demonstration of Nileprost Inhibition of Cytotoxic CD8+ Lymphocyte Cytokine Release

### Principle:

The activation of a human T lymphocyte by an antigen-presenting cell and antigen through the T cell receptor is mimicked in experimental conditions by the addition of antibodies to the CD3 subunit of the T cell receptor and antibodies to the CD28 costimulatory receptor. It is known that anti-CD3 anti-CD28 binding to T lymphocytes stimulates the cells to release various cytokines. Some of these cytokines are IL-2, IFN-γ and GM-CSF. The biochemistry and biological activities of these cytokines have been extensively reviewed in the literature. The binding of Nileprost to the EP receptor inhibits the release of various CD8+ cytokines.

### Detection Method:

Multi-cytokine immunosorbant assays based on antibodies specific to human cytokines are commercially available. Standards and samples are pipetted into sample tubes. A monoclonal antibody specific for a cytokine is covalently linked to a fluorescent bead set, which captures the cytokine. A complementary biotinylated monoclonal cytokine antibody then completes the immunological sandwich and the reaction is detected with streptavidin-phycoerythrin.

### Procedure:

CD14-negative populations were isolated from four donors by Miltenyi CD14 beads. The four populations were allowed to rest at 5×10⁶/mL in separate flasks overnight in RPMI 1640, 10% fetal bovine serum. Meanwhile, anti-CD3 antibody (OKT3, functional antibody, eBioscience) were bound to 10 cm plates at 5 μg/mL in sodium carbonate binding buffer at 4° C. The next day, the cells were mixed and 1×10⁹ cells were taken for CD8 isolation using the Milenyi CD8 isolation kit (negative selection). The resulting 3.9×10⁸ CD8 cells were resuspended in medium at 4×10⁶/mL and added to the 10 cm CD3-bound plates. Soluble anti-CD28 (2-5 μg/mL) and 1 μM Nileprost or vehicle alone were added to the CD8 cells. Incubation was continued overnight and cytokines were detected as described above. The results are presented below.

Nileprost Inhibition (percent)

| | |
|---|---|
| Nileprost | standard deviation |

human CD8 cells
Anti-CD3/CD28 stimulation
IFN-γ (pg/mL)

| | | |
|---|---|---|
| Donor 1 | 79% | 1% |
| Donor 2 | 59% | 1% |
| Donor 3 | 79% | 2% |
| Donor 4 | 80% | 1% |

human CD8 cells
Anti-CD3/CD28 stimulation
GM-CSF (pg/mL)

| | | |
|---|---|---|
| Donor 1 | 77% | 0% |
| Donor 2 | 65% | 2% |
| Donor 3 | 87% | 1% |
| Donor 4 | 61% | 6% |

human CD8 cells
Anti-CD3/CD28 stimulation
IL-2 (pg/mL)

| | | |
|---|---|---|
| Donor 1 | 71% | 0% |
| Donor 2 | 68% | 4% |
| Donor 3 | 88% | 0% |
| Donor 4 | 70% | 5% |

### Nileprost showed very high inhibitory activity in the cytotoxic CD8+ lymphocyte cytokine release.

### Example 3

### Demonstration of Nileprost Inhibiton of Human Monocyte-Derived Dendritic Cell (DC) Cytokine Release

### Principle:

Dendritic cells (DCs) are the most potent antigen-presenting cells and play a central role in immune response. Following stimulation through the toll-like receptors TLR, DCs express and release proinflammatory cytokines and chemokines and may induce activation and proliferation of naïve T cells. The binding of Nileprost to the EP receptor inhibits TLR4 ligand (LPS)-stimulated IL-12 release. Therefore, Nileprost skews the CD4 T cell differentiation to a Th-2 lineage.

### Detection Method:

IL-12 is a 75 kDa glycoprotein heterodimer (p70) composed of two genetically unrelated subunits linked by a disulfide bond. Enzyme-linked immunosorbant assays based on antibodies specific to IL-12 p70 are commercially available. Standards and samples are pipetted into the wells of a microplate. An antibody specific to human IL-12 is added to the wells. A substrate is added to the wells and color develops in proportion to the amount of IL-12 bound. The intensity of the color is measured.

### Procedure:

Human monocyte-derived dendritic cells were isolated from human donors using a Ficoll density gradient and residual erythrocytes were removed by selective lysis. CD14 MicroBeads were used for separation of human cells based on the expression of the CD14 antigen. The dendritic cells were cultured at approximately 1.5×10⁶ cells per mL in RPMI1640 with fetal bovine serum, 200 ng/mL GM-CSF (Leukine) and 10 ng/mL IL-4. The cells grew for a period of 3 days and then the media was changed. 10 ng/mL LPS was used to activate the cells. 1 μM of Nileprost and 1 M of PGE₂ were added during the LPS stimulation. Cells were incubated for approximately 18 hr at 37° C. IL-12 released during activation was measured by ELISA. The results are presented below.

Nileprost Inhibition (percent)
Human monocyte derived dendritic cells
LPS stimulation
IL-12 (pg/mL)

| | Nileprost | PGE2 |
|---|---|---|
| Donor 1 | 84% | 80% |
| Donor 2 | 51% | 33% |
| Donor 3 | 69% | 83% |
| Donor 4 | 66% | 72% |

Nileprost showed very high inhibitory activity in human monocyte-derived dendritic cell (DC) cytokine release.

What is claimed is:

1. A method of treating an autoimmune disease in a patient in need of such treatment comprising administering to the patient a therapeutically effective amount of a 5-cyano-prostacyclin derivative or a pharmaceutically acceptable salt or cyclodextrin clathrate thereof.

2. A method according to claim 1, wherein said 5-cyano-prostacyclin derivative is Nileprost.

3. A method according to claim 1, wherein said autoimmune disease is selected from the group consisting of multiple sclerosis, secondary progressive multiple sclerosis, psoriasis, rheumatoid arthritis, Crohn's disease, and alopecia areata.

4. A method according to claim 1, wherein said autoimmune disease is multiple sclerosis.

5. A method according to claim 2, wherein said autoimmune disease is selected from the group consisting of multiple sclerosis, secondary progressive multiple sclerosis, psoriasis, rheumatoid arthritis, Crohn's disease, and alopecia areata.

6. A method according to claim 2, wherein said autoimmune disease is multiple sclerosis.

7. A method according to claim 1, wherein said 5-cyano-prostacyclin derivative is administered as a salt or a cyclodextrin clathrate.

8. A method according to claim 2, wherein said Nileprost is administered as a salt or a cyclodextrin clathrate.
